(19) United States
(12) Patent Application Publication
(10) Pub. No.: US 2024/0366631 A1
(43) Pub. Date: Nov. 7, 2024

(54) METHOD OF TREATING OPERATIVE COMPLICATIONS OF CATARACT SURGERY

(71) Applicant: FORMOSA PHARMACEUTICALS, INC., Taipei City (TW)

(72) Inventors: Chen Yu CHENG, Taipei City (TW); Laurene WANG, Chapel Hill, NC (US); Wei-Cheng LIAW, San Diego, CA (US); Derek Joseph Raphael NUNEZ, Raleigh, NC (US)

(73) Assignee: FORMOSA PHARMACEUTICALS, INC., Taipei City (TW)

(21) Appl. No.: 18/310,036

(22) Filed: May 1, 2023

(51) Int. Cl. A61K 31/57; A61K 9/10; A61K 9/51; A61P 27/12

(52) U.S. Cl. CPC A61K 31/57; A61K 9/10; A61K 9/5115; A61K 9/5123; A61K 9/5138; A61K 9/5161; A61P 27/12

(57) ABSTRACT

The present disclosure provides a method of treating or ameliorating an operative complication of a cataract surgery. The method comprises administering nanoparticles of clobetasol propionate to a subject in need thereof.

### FIELD OF THE INVENTION

The present disclosure relates to a therapeutic method, more particularly to a method of treating an operative complication of cataract surgery.

### BACKGROUND OF THE INVENTION

A “cataract” is a clouding of the lens in the eye that affects vision. Most people develop cataracts due to aging. Cataract surgery is one of the most common eye surgeries. The main purpose of cataract surgery is to remove the cloudy crystals, implant artificial crystals or intraocular lenses (IOL), and restore some of the optical properties of the cloudy crystals.

With new advancements in technology, infectious disease control, and equipment, there are fewer postoperative adverse events following the procedure. However, complications still occur post-operatively.

There is thus a need for improved methods of treating an operative complication of cataract surgery.

### SUMMARY OF THE INVENTION

The present disclosure provides a novel method of treating or ameliorating an operative complication of cataract surgery.

In one embodiment of the present disclosure, the method of treating or ameliorating an operative complication of cataract surgery in a subject in need thereof comprises administering a pharmaceutical composition comprising an aqueous suspension to a subject in need thereof in a therapeutically effective amount, wherein the aqueous suspension comprises: nanoparticles of clobetasol propionate, wherein a mean particle diameter of the nanoparticles is 300 nm or less and a D90 particle diameter of the nanoparticles is 450 nm or less;
a physiologically acceptable salt;
glycerin;
hydrogenated soybean lecithin; and
anhydrous citric acid.

Examples of the operative complication include but are not limited to high anterior chamber cell (ACC) count, pain, impaired visual acuity, ocular inflammation and ocular infection.

In some embodiments of the disclosure, the pharmaceutical composition is administered before the cataract surgery, in progress of the cataract surgery or after the cataract surgery. In one embodiment of the disclosure, wherein the pharmaceutical composition is administered after the cataract surgery.

In one embodiment of the disclosure, the cataract surgery is an uncomplicated cataract surgery or complicated cataract.

In some embodiments of the disclosure, the aqueous suspension comprises 0.01 wt % to 0.2 wt %; 0.015 wt % to 0.18 wt %; 0.02 wt % to 0.16 wt %; 0.025 wt % to 0.14 wt %; 0.03 wt % to 0.12 wt %; 0.035 wt % to 0.1 wt %; 0.04 wt % to 0.08 wt %; 0.04 wt % to 0.06 wt %; 0.05% wt; 0.1% wt % of clobetasol propionate.

In one embodiment of the disclosure, the pharmaceutical composition is administered for at least once.

In one embodiment of the disclosure, the pharmaceutical composition is administered once, twice, three times, or four times a day.

In one embodiment of the disclosure, the pharmaceutical composition is administered on the first post-operative day (POD 1).

In one embodiment of the disclosure, the pharmaceutical composition is administered for at least one day. In one embodiment of the disclosure, the pharmaceutical composition is administered for 3 days to 28 days; 4 days to 27 days; 5 days to 26 days; 6 days to 26 days; 7 days to 25 days; 8 days to 24 days; 9 days to 23 days; 10 days to 22 days; 11 days to 21 days; 12 days to 20 days; 13 days to 19 days; 14 days to 18 days; 15 days to 17 days.

In some embodiments of the disclosure, the nanoparticles are produced by mixing clobetasol propionate, the physiologically acceptable salt, glycerin, hydrogenated soybean lecithin and anhydrous citric acid.

In some embodiments of the disclosure, the nanoparticles are produced by further mixing clobetasol propionate, the physiologically acceptable salt, glycerin, hydrogenated soybean lecithin and anhydrous citric acid with a surface modifier.

In some embodiments of the disclosure, the surface modifier comprises a surfactant, agglomeration inhibitor, viscosity modifier and/or dispersion stabilizer.

In some embodiments of the disclosure, the surface modifier is the dispersion stabilizer.

Examples of the dispersion stabilizer include but are not limited to polyoxyethylene polyoxypropylene glycol and/or polyvinyl alcohol.

In some embodiments of the disclosure, the surface modifier is the viscosity modifier.

Examples of the viscosity modifier include but are not limited to methyl cellulose, hydroxypropyl methylcellulose and polyvinyl alcohol.

In some embodiments of the disclosure, the aqueous suspension comprises 1 to 10 mg/mL; 1.5 to 9.5 mg/mL; 2 to 9 mg/mL; 2.5 to 8.5 mg/mL; 3 to 8 mg/mL; 3.5 to 7.5 mg/mL; 4 to 7 mg/mL; 4.5 to 6.5 mg/mL; 5 to 6 mg/mL of the viscosity modifier.

In some embodiments of disclosure, the pharmaceutical composition is administered as an eye drop.

The present disclosure provides an ophthalmic composition comprising:
nanoparticles of clobetasol propionate, wherein a mean particle diameter of the nanoparticles is 300 nm or less and a D90 particle diameter of the nanoparticles is 450 nm or less;
a physiologically acceptable salt;
glycerin;
hydrogenated soybean lecithin;
anhydrous citric acid;
boric acid;
ethylenediaminetetraacetic acid (EDTA); and
benzalkonium chloride (BAK), wherein the concentration of benzalkonium chloride is lower than 45 ppm.

In some embodiments of the disclosure, the ophthalmic composition comprises the nanoparticles of clobetasol propionate, sodium chloride, hydrogenated soybean lecithin, glycerin, anhydrous citric acid, polyvinyl alcohol, Poloxamer 407, BAK, methylcellulose, boric acid, and EDTA.

In some embodiments of the disclosure, the ophthalmic composition comprises 0.05 wt % to 0.15 wt % of the nanoparticles of clobetasol propionate, 0.5 wt % to 1.5 wt % sodium chloride, 0.05 wt % to 0.15 wt % hydrogenated soybean lecithin, 0.1 wt % to 0.4 wt % glycerin, 0.003 wt % to 0.01 wt % anhydrous citric acid, 0.05 wt % to 0.15 wt % polyvinyl alcohol, 0.002 wt % to 0.01 wt % Poloxamer 407, 0.003 wt % to 0.004 wt % BAK. 0.3 wt % to 0.8 wt % methylcellulose, 0.15 wt % to 0.35 wt % boric acid, and 0.01 wt % to 0.07 wt % EDTA.

These and other aspects will become apparent from the following description of the preferred embodiments taken in conjunction with the following drawings, although variations and modifications may be effected without departing from the spirit and scope of the novel concepts of the disclosure.

### BRIEF DESCRIPTION OF THE DRAWINGS

FIG. 1 shows the results of mean anterior chamber cell counts treated with Suspensions 1 to 2 and Placebo Suspensions 1 to 2 after cataract surgery.

FIG. 2 shows the percentage of subjects with an ACC count of 0 at a given time point treated with Suspensions 1 to 2 and Placebo Suspensions 1 to 2 after cataract surgery.

FIG. 3 shows the percentage of the subjects with a pain index of 0 at each time point treated with Suspensions 1 to 2 and Placebo Suspensions 1 to 2 after cataract surgery.

### DETAILED DESCRIPTION OF THE INVENTION

### Definitions

Unless otherwise defined, all technical and scientific terms used herein have the same meaning as commonly understood by one of ordinary skill in the art to which this disclosure pertains. In the case of conflict, the present document, including definitions, will control. As used in this specification and the appended claims, the singular forms “a,” “an” and “the” include plural references unless the content clearly dictates otherwise.

Throughout this specification, unless the context requires otherwise, the word “comprise,” or variations such as “comprises” or “comprising.” will be understood to imply the inclusion of a stated element or integer or group of elements or integers but not the exclusion of any other element or integer or group of elements or integers.

As used herein, the terms “treatment” and “treating” embrace both preventative, i.e. prophylactic, or therapeutic, i.e. curative and/or palliative, treatment. Thus, the terms “treatment” and “treating” comprise therapeutic treatment of patients having already developed said condition, particularly in manifest form. Therapeutic treatment may be symptomatic treatment in order to relieve the symptoms of the specific indication or causal treatment in order to reverse or partially reverse the conditions of the indication or to stop or slow down the progression of the disease. Thus, the conjugates, compositions, and methods of the present disclosure may be used for instance as therapeutic treatment over a period of time as well as for chronic therapy. In addition, the terms “treatment” and “treating” comprise prophylactic treatment, i.e. a treatment of patients at risk to develop a condition mentioned hereinbefore, thus reducing said risk.

As used herein, the term “therapeutically effective amount” means an amount of a conjugate of the present disclosure that (i) treats or prevents the particular disease or condition, (ii) attenuates, ameliorates, or eliminates one or more symptoms of the particular disease or condition, or (iii) prevents or delays the onset of one or more symptoms of the particular disease or condition described herein.

As interchangeably used herein, the terms “individual,” “subject,” “host,” and “patient,” refer to a mammal, including, but not limited to, murines (rats, mice), non-human primates, humans, canines, felines, ungulates (e.g., equines, bovines, ovines, porcines, caprines), etc.

As used herein, the term “in need of treatment” refers to a judgment made by a caregiver (e.g., physician, nurse, nurse practitioner, or individual in the case of humans; veterinarian in the case of animals, including non-human mammals) that a subject requires or will benefit from treatment. This judgment is made based on a variety of factors that are in the realm of a caregiver's expertise, but that includes the knowledge that the subject is ill, or will be ill, as the result of a condition that is treatable by the compounds of the present disclosure.

### Aqueous Suspension Containing Nanoparticles of Clobetasol Propionate

Particularly, the inventors found that nanoparticles of clobetasol propionate have excellent transferability into the aqueous humor and good anti-inflammatory action, when the nanoparticles have a mean particle diameter of 300 nm or less and a 90% diameter (hereinafter referred to as “D90”) of 450 nm or less (preferably, a mean particle diameter of 250 nm or less and a D90 of 300 nm or less, or a mean particle diameter of 200 nm or less and a D90 of 250 nm or less). When such nanoparticles are employed, the solubility of clobetasol propionate is expected to become higher, which increases the bioavailability and reduces the required administration dose. The mean particle diameter can be measured as number distribution mean particle diameter.

The mean particle diameter ranges of the pulverized clobetasol propionate particles obtained by the above production method can be the same as the mean particle diameter of the clobetasol propionate nanoparticles contained in the aqueous suspension or aqueous pharmaceutical composition described above. Also, the ranges of the 90% diameter (D90) and the 50% diameter (D50) of the pulverized clobetasol propionate particles obtained by the above production method can be the same as the 90% diameter (D90) and the 50% diameter (D50), respectively, of the clobetasol propionate nanoparticles contained in the aqueous suspension or aqueous pharmaceutical composition described above.

The “mean particle diameter” herein means the arithmetic mean diameter of the particle size distribution measured by dynamic light scattering photon correlation spectroscopy. The 50% diameter (also referred to as median diameter, D50) represents the diameter at which powder particles are divided into two groups in the particle size distribution measured by the above measurement method, wherein the amounts of particles are equal between said two groups, the larger diameter group and the smaller diameter group. The “90% diameter” means the diameter (D90) of the particle at 90% position in the particle size distribution measured by the above measurement method, wherein the number of particles is counted from the smaller particle diameter to the larger particle diameter, as setting 0% (less smallest) to 100% (the largest particle). The “10% diameter” means the diameter (D10) of the particle at 10% position in the particle size distribution measured by the above measurement method, wherein the number of particles is counted from the smaller particle diameter to the larger particle diameter, as setting 0% (less smallest) to 100% (the largest particle). The measurement method by dynamic light scattering photon correlation spectroscopy, and the calculation method of particle size distribution are well known in the art.

The present disclosure thus relates to, in one embodiment, the aqueous suspension containing nanoparticles of clobetasol propionate, and preferably to the aqueous suspension wherein the nanoparticles has a mean particle diameter of 300 nm or less and a D90 of 450 nm or less. The aqueous suspension contains, for example, nanoparticles of clobetasol propionate produced by mixing clobetasol propionate, a physiologically acceptable salt, a physiologically acceptable polyol and/or water and a surface modifier. Examples of the surface modifier include, but are not limited to a surfactant, agglomeration inhibitor, viscosity modifier and dispersion stabilizer. The aqueous suspension more preferably contains nanoparticles of clobetasol propionate produced by mixing clobetasol propionate, a physiologically acceptable salt, glycerin, anhydrous citric acid and hydrogenated soybean lecithin.

The inventors additionally found that the aqueous suspension containing nanoparticles of clobetasol propionate, polyoxyethylene polyoxypropylene glycols (hereinafter referred to as “POE-POP glycol”) and/or polyvinyl alcohols (hereinafter referred to as “PVA”) as a dispersion stabilizer, and/or hydroxypropyl methylcellulose and/or methyl cellulose as a viscosity modifier exhibits good long-term transparency, dispersibility, and storage stability.

The present disclosure thus relates to, in one embodiment, the aqueous suspension containing nanoparticles of clobetasol propionate having a mean particle diameter of 300 nm or less and a D90 of 450 nm or less (preferably, a mean particle diameter is 250 nm or less and a D90 is 300 nm or less, or a mean particle diameter is 200 nm or less and a D90 is 250 nm or less). The present disclosure relates to, in another embodiment, the aqueous pharmaceutical composition containing nanoparticles of clobetasol propionate as an effective agent and a dispersion stabilizer and/or a viscosity modifier as an additive.

The “aqueous pharmaceutical composition” herein means an aqueous liquid or gel pharmaceutical composition, specifically a pharmaceutical composition containing nanoparticles of clobetasol propionate suspended in the aqueous liquid or gel. The pharmaceutical composition herein accordingly means an aqueous pharmaceutical composition unless otherwise stated. The aqueous pharmaceutical composition includes topical preparations. The topical preparations herein accordingly mean aqueous preparations for topical administration. The aqueous pharmaceutical composition may be viscous as long as it doesn't prevent the composition from being used as a pharmaceutical drug, and includes gel preparations as well as watery preparations.

The “topical area” herein means a part of the body, including an affected site, an area around the affected site or an organ including the affected site, and preferably is the eye.

The “topical preparation” herein means a pharmaceutical composition to be administered locally. The topical preparation preferably includes topical eye preparations (e.g., eye drops).

The aqueous pharmaceutical composition can be used to treat or prevent an operative complication of a cataract surgery by topically administering an effective amount thereof to a patient in need thereof. In other words, the present disclosure relates to, in one embodiment, a method for treatment or prevention of inflammatory or infectious diseases comprising administering a therapeutically effective amount of the aqueous suspension or the pharmaceutical composition containing the aqueous suspension, wherein the aqueous suspension or pharmaceutical composition contains nanoparticles of clobetasol propionate and optionally a dispersion stabilizer and/or a viscosity modifier to a patient in need thereof.

Alternatively, the present disclosure relates to a use of nanoparticles of clobetasol propionate (optionally with a surface modifier, surfactant, agglomeration inhibitor, dispersion stabilizer and/or a viscosity modifier) or a use of an aqueous suspension containing said nanoparticles, for manufacturing an aqueous pharmaceutical composition (e.g., injections and topical preparations).

The “aqueous suspension” herein means an aqueous liquid in which nanoparticles of clobetasol propionate are suspended. The aqueous suspension herein may constitute a pharmaceutical composition which can be administered as a pharmaceutical drug by itself, or may constitute a pharmaceutical composition by adding other components and a diluent (e.g. raw materials for pharmaceutical composition), or may not be used for a pharmaceutical drug.

The aqueous suspension herein includes dispersion-stabilized aqueous suspensions. The dispersion-stabilized means that the aqueous suspension has any one of, or two or more of, the properties of (1) no precipitation confirmed under visual inspection, (2) high transparency, (3) no agglomerate or crystal observed under microscopic observation, and (4) no substantial changes in the mean particle diameter value (no more than 50%) after dispersion by stirring followed by standing for 24 hours (preferably 2 days, 3 days, 4 days, 5 days, 6 days, 7 days, 1 month, 2 months, 3 months, 4 months, 5 months, 6 months, 1 year or 2 years) at room temperature (25° C.). The aqueous suspension containing nanoparticles of clobetasol propionate herein is preferably an aqueous suspension with no precipitation confirmed under visual inspection, high transparency, and no agglomerate or crystal observed under microscopic observation after 7 days from being sealed in a test tube.

The transparency can be determined in conformity with the transparency test described in The Japanese Pharmacopoeia. Specifically, the transparency can be determined by the following procedures. Water is added to 5 mL of a formazine standard up to 100 mL, which is used as a turbidity standard. Each of a tested aqueous suspension and a newly prepared turbidity standard is taken to a colorless clear glass flat-bottom test tube having an inner diameter of 15 mm such that the liquid layer has a depth of 30 mm or 40 mm, which is then compared with the others by observation from above on a black backdrop in the scattering light. When the transparency of the tested aqueous suspension is the same as water or the solvent used, or when the turbidity of the tested aqueous suspension is lower than the turbidity standard, the transparency is determined to be high. Alternatively, transmittances at 660 nm of a tested aqueous suspension and of a newly prepared turbidity standard are measured by the ultraviolet visible spectrophotometry method using a 50 mm layer cell, with water or the solvent as the control. When the transmittance of the tested aqueous suspension is higher than the turbidity standard, the transparency of the tested aqueous suspension is determined to be high.

In another embodiment, the topical preparation is a topical eye preparation having transferability into the aqueous humor. The “transferability into the aqueous humor” herein may mean that a concentration of clobetasol propionate (average value) in the aqueous humor is 45 ng/ml or more (preferably 50 ng/ml or more, 55 ng/ml or more, 60 ng/ml or more, 65 ng/ml or more, 70 ng/ml or more, 75 ng/ml or more) after 60 minutes from a single eye drop administration of the aqueous topical preparation containing clobetasol propionate adjusted to be 0.05% (w/v). The “transferability into the aqueous humor” may alternatively mean that a concentration of clobetasol propionate (average value) in the aqueous humor is 40 ng/ml or more (preferably 50 ng/ml or more, 55 ng/ml or more, 60 ng/mL or more, 63 ng/ml or more, 64 ng/ml or more, 65 ng/mL or more, 70 ng/ml or more, 75 ng/ml or more) after 30 minutes from a single eye drop administration of the aqueous topical preparation containing clobetasol propionate adjusted to be 0.05% (w/v).

In another embodiment, the topical preparation is a topical eye preparation having transferability into the conjunctiva. The “transferability into the conjunctiva” herein means that a concentration of clobetasol propionate (average value) in the conjunctiva is 500 ng/ml or more (preferably 659 ng/ml or more, 900 ng/ml or more, 972 ng/mL or more, 1000 ng/ml or more, 1200 ng/ml or more, 1210 ng/mL or more, 1400 ng/ml or more, 1455 ng/ml or more, 1500 ng/ml or more or 2000 ng/ml or more, 2141 ng/mL or more) after 15 minutes from a single eye drop administration of the aqueous topical preparation containing clobetasol propionate adjusted to be 0.05% (w/v).

The transferability into the aqueous humor and the conjunctiva can be determined according to the method described in the Examples of this application by using appropriate animals, and for example by the following procedures. The lower eyelid of a rabbit is gently pulled off, an eye drop of the test substance is administered (a single eye drop administration) into the conjunctival sac of the left eye using a pipette, and after administration, the upper and lower eyelids are gently closed and held for about 2 seconds. After 15 minutes, 30 minutes, 60 minutes and 90 minutes from the administration, the rabbits are anesthetized and euthanized by bleeding, followed by thoroughly washing the eye with water for injection, and the aqueous humor and conjunctiva are collected. A concentration of clobetasol propionate in the collected aqueous humor can be determined by adding methanol and an internal standard (prednisolone) solution to the collected aqueous humor, stirring the mixture, subsequently adding acetonitrile thereto, stirring the mixture, and centrifuging (13,100×g, 4° C., 5 minutes) the mixture, followed by measuring the supernatant obtained by centrifuge by the LC-MS/MS method. A concentration of clobetasol propionate in the collected conjunctiva can be determined by adding ultrapure water in nine fold volume of the wet weight of the obtained conjunctiva, homogenizing, further adding methanol and an internal standard (prednisolone) solution thereto, stirring the mixture, subsequently adding acetonitrile thereto, stirring the mixture, and centrifuging the mixture (13100×g, 4° C., 5 minutes), followed by measuring the supernatant obtained by centrifuge by the LC-MS/MS method.

In another embodiment, the topical preparation is a topical eye preparation capable of reducing an increase rate of protein concentration in the aqueous humor. Being “capable of reducing an increase rate of protein concentration in the aqueous humor” means that a protein concentration in the aqueous humor which is obtained by administering 40 μL of the aqueous topical preparation containing a 0.05% (w/v) or 0.1% (w/v) clobetasol propionate seven times at 30-60 minutes intervals before and after keratocentesis (preferably, setting the time of keratocentesis as 0 minutes, seven administrations at 180 minutes, 120 minutes, 60 minutes and 30 minutes before the keratocentesis, and 30 minutes, 60 minutes and 90 minutes after the keratocentesis) to an experimental animal (e.g., rabbit) and collecting the aqueous humor after 30 minutes from the final administration, is less than three times (preferably less than 2.5 times or less than two times) the protein concentration in the aqueous humor of the eye on which keratocentesis is not carried out.

The topical eye preparation may have two or more (two, three or all) properties selected from the transferability into the aqueous humor, the transferability into the conjunctiva, the reduction of increasing rate of a protein concentration in the aqueous humor and the inflammation inhibitory activity on the eye.

In one embodiment, the aqueous suspension is an aqueous suspension with low irritability. The low irritability herein means that a degree of irritating reactions (inflammation reactions such as flare, swelling and/or congestion) in administering the aqueous suspension to a subject is lower than that in administering previously used aqueous preparations containing the same active component. Whether the irritability of a test aqueous suspension is low or not can be determined, for example, with reference to the method of Jonas, J. Kuchne et al., Am J Ophthalmol (2004) 138:547-553, by administering the test aqueous suspension to the eye of a rabbit, measuring the degree of eye inflammation, and determining that the irritability is low when the degree of inflammation is lower than the standard liquid agent (the same as above). More specifically, in the case of an eye drop, the irritability is determined by applying a preparation containing 1.0% clobetasol propionate to the eye once to 20 times a day at intervals of 30 minutes to several hours, observing the cornea, iris and conjunctiva before administration and 1, 3, 5 and 24 hours after the final administration, and scoring in accordance with Draize's scoring criteria (see OECD GUIDELINES FOR TESTING OF CHEMICALS 405 (24 Feb. 1987) Acute Eye Irritation/Corrosion).

The aqueous suspension or pharmaceutical composition may contain one or two or more physiologically acceptable salts. Examples of the “physiologically acceptable salt” include sodium chloride, potassium chloride, ammonium chloride, sodium sulfate, magnesium sulfate, potassium sulfate, calcium sulfate, sodium malate, sodium citrate, disodium citrate, sodium dihydrogen citrate, potassium dihydrogen citrate, sodium dihydrogen phosphate, potassium dihydrogen phosphate, disodium hydrogen phosphate, and dipotassium hydrogen phosphate, and sodium chloride is preferable.

The aqueous suspension or pharmaceutical composition can contain the physiologically acceptable salt at a concentration of 0.01 to 10%, preferably 0.1 to 5% or, for example, 0.5 to 3%, and 0.8 to 2%. Alternatively, the aqueous suspension or pharmaceutical composition can contain the physiologically acceptable salt at a concentration of 0.01 to 50 mg/mL, 0.1 to 20 mg/mL or 1 to 5 mg/mL.

The aqueous suspension or pharmaceutical composition may contain one or two or more surfactants and/or one or two or more agglomeration inhibitors.

The “surfactant” is not limited as long as it can be administered to a human as a pharmaceutical additive without showing toxicity and without hindering the activity of clobetasol propionate. The surfactant may be, for example, non-ionic surfactant including polyoxyethylene (hereinafter referred to as “POE”)-polyoxypropylene (hereinafter referred to as “POP”) block copolymers such as poloxamer 407, poloxamer 235 and poloxamer 188; ethylenediamine adducts to polyoxyethylene-polyoxypropylene block copolymer such as poloxamine; POE sorbitan fatty acid esters such as POE (20) sorbitan monolaurate (polysorbate 20), POE (20) sorbitan monooleate (polysorbate 80) and polysorbate 60; POE hydrogenated castor oils such as POE (60) hydrogenated castor oil; POE alkyl ethers such as POE (9) lauryl ether; POE-POP alkyl ethers such as POE (20) POP (4) cetyl ether; POE alkylphenyl ethers such as POE (10) nonyl phenyl ether; POE-POP glycols such as POE (105) POP (5) glycol. POE (120) POP (40) glycol, POE (160) POP (30) glycol, POE (20) POP (20) glycol, POE (200) POP (70) glycol, POE (3) POP (17) glycol, POE (42) POP (67) glycol, POE (54) POP (39) glycol and POE (196) POP (67) glycol; amphoteric surfactants including glycine-type surfactants such as alkyldiaminoethyl glycine, betaine acetate-type surfactants such as lauryl dimethylaminoacetic acid betaine, and imidazoline-type surfactants; anionic surfactants including POE alkyl ether phosphates and salts thereof such as POE (10) sodium lauryl ether phosphate, N-acylamino acid salts such as sodium lauroyl methyl alanine, alkyl ether carboxylates, N-acyl taurates such as sodium cocoyl N-methyltaurate, sulfonates such as sodium tetradecenesulfonate, alkyl sulfates such as sodium lauryl sulfate, POE alkyl ether sulfates such as POE (3) sodium lauryl ether sulfate, and α-olefin sulfonates; and cationic surfactants including alkylamine salts, alkyl quarternary ammonium salts (benzalkonium chloride and benzethonium chloride) and alkyl pyridinium salts (cetylpyridinium chloride and cetylpyridinium bromide). The aqueous suspension may contain one or two or more surfactants.

The “agglomeration inhibitor” herein is not limited as long as it inhibits an agglomeration of clobetasol propionate and it can be administered to a human without showing toxicity and without hindering the activity of clobetasol propionate. The agglomeration inhibitor may be phospholipids such as alkyl sulfate, N-alkyloyl methyl taurate, ethanol, glycerol, propylene glycol, sodium citrate, phospholipids including glycerophospholipid (lecithin (phosphatidylcholine) (e.g., refined soybean lecithin, hydrogenated soybean lecithin), phosphatidylserine, phosphatidylethanolamine, phosphatidylinositol, phosphatidic acid, phosphatidylglycerol, lysophosphatidylcholine, lysophosphatidylserine, lysophosphatidylethanolamine, lysophosphatidylinositol, lysophosphatidic acid and lysophosphatidylglycerol) and sphingophospholipids (sphingomyelin, ceramide, glycosphingolipid or ganglioside), D-sorbitol, lactose, xylitol, gum arabic, sucrose fatty acid ester, polyoxyethylene hydrogenated castor oil, polyoxyethylene fatty acid esters, polyethyleneglycol (PEG), polyoxyethylene sorbitan fatty acid ester, alkyl benzene sulfonate, sulfosuccinate, POE-POP glycol, polyvinylpyrrolidone, PVA, hydroxypropyl cellulose, methyl cellulose, hydroxyethyl cellulose, hydroxypropyl methylcellulose, carmellose sodium, carboxyvinyl polymers, N-acyl-glutamate, acrylic acid copolymers, methacrylic acid copolymers, casein sodium, L-valine, L-leucine, L-isoleucine, benzalkonium chloride and benzethonium chloride. The aqueous suspension may contain one agglomeration inhibitor or two or more agglomeration inhibitors.

The aqueous suspension or pharmaceutical composition can contain agglomeration inhibitor at a concentration of 0.001 to 10% or 0.01 to 10%, preferably 0.02 to 5%, for example, 0.03 to 1%, 0.04 to 0.5%, 0.05 to 0.2%. Alternatively, the aqueous suspension or pharmaceutical composition can contain the agglomeration inhibitor at a concentration of 0.01 to 50 mg/mL, 0.1 to 20 mg/mL or 1 to 5 mg/mL.

The surfactant and/or the agglomeration inhibitor are preferably one or more substances selected from polyoxyethylene hydrogenated castor oil 60 (e.g., HCO-60), polyoxyethylene hydrogenated castor oil 40 (e.g., HCO-40), polysorbate 80 (e.g., Tween 80), polysorbate 20 (e.g., Tween 20), POE-POP glycol (e.g., PLONON 407P, Pluronic F68, UNILUBE 70DP-950B) and PVA (e.g., Kuraray POVAL 217C), and more preferably one or more substances selected from POE-POP glycol and PVA.

The “viscosity modifier” herein is not limited as long as it is capable of adjusting the viscosity of the aqueous suspension and can be administered to a human as a pharmaceutical additive without showing toxicity and without hindering the activity of clobetasol propionate. The viscosity modifier may be polysaccharides or derivatives thereof (gum arabic, gum karaya, xanthan gum, carob gum, guar gum, gum guaiac, quince seed, darman gum, gum tragacanth, benzoin rubber, locust bean gum, casein, agar, alginic acid, dextrin, dextran, carrageenan, gelatin, collagen, pectin, starch, polygalacturonic acid, chitin and derivatives thereof, chitosan and derivatives thereof, elastin, heparin, heparinoid, heparin sulfate, heparan sulfate, hyaluronic acid and chondroitin sulfate), ceramide, cellulose derivatives (methyl cellulose, ethyl cellulose, hydroxyethyl cellulose, hydroxypropyl cellulose, hydroxypropyl methylcellulose, carboxymethyl cellulose, carboxyethyl cellulose, cellulose and nitrocellulose), PVA (completely or partially saponified), polyvinylpyrrolidone, Macrogol, polyvinyl methacrylate, polyacrylic acid, carboxyvinyl polymer, polyethyleneimine, polyethylene oxide, polyethylene glycol, ribonucleic acid, deoxyribonucleic acid, methyl vinyl ether-maleic anhydride copolymers, and pharmacologically acceptable salts thereof (e.g., sodium alginate). The aqueous suspension may contain one or two or more viscosity modifiers. The viscosity modifier is preferably one or more substances selected from hydroxypropyl methylcellulose (e.g., TC-5(R), Metlose 60SH-50), PVA (Kurary POVAL 217C) and methyl cellulose (e.g., Metlose SM-100, Metlose SM-15), with one or more substances selected from hydroxypropyl methylcellulose and methyl cellulose being more preferable.

The aqueous suspension can contain 1 to 10 mg/mL, preferably 1 to 5 mg/mL, for example, 1 to 4 mg/mL, 1 to 3 mg/mL, 1 to 2 mg/mL, of the viscosity modifier.

The “surface modifier” herein refers to the surfactant, the agglomeration inhibitor, the viscosity modifier and/or the dispersion stabilizer, and the surface modifier is capable of modifying the nanoparticle surface of clobetasol propionate.

The “dispersion stabilizer” usable herein is listed above as the surfactants, agglomeration inhibitors and/or viscosity modifiers, and is preferably one or more substances selected from polyoxyethylene hydrogenated castor oil 60, polyoxyethylene hydrogenated castor oil 40, polysorbate 80, polysorbate 20, POE-POP glycol, PVA, hydroxypropyl methylcellulose and methyl cellulose, and more preferably one or more substances selected from POE-POP glycol, PVA, hydroxypropyl methylcellulose and methyl cellulose.

The surfactant, agglomeration inhibitor and/or viscosity modifier which are also used as the dispersion stabilizer (hereinafter referred to as “additives” in this paragraph) may adhere to or be adsorbed on the surface of nanoparticles of clobetasol propionate. When these additives are added before the pulverization step, these additives adhere to or are adsorbed on the surface of nanoparticles of clobetasol propionate, which results in inhibiting the nanoparticle agglomeration during the pulverization step. By adhering to or by adsorbing on the surface of nanoparticles of clobetasol propionate, the additives effectively inhibit the agglomeration in the aqueous suspension. In this context, the surfactant, agglomeration inhibitor and/or viscosity modifier which can be also used as the dispersion stabilizer can be construed to adhere to or to be adsorbed on the surface of nanoparticles of clobetasol propionate, as long as at least a part of the additives adheres to or is adsorbed on the nanoparticle surface (contributing to the surface modification), and it is not necessary that the additive is adhered or adsorbed in the aqueous suspension.

The aqueous suspension or pharmaceutical composition may contain one or two or more physiologically acceptable polyols. The pharmaceutical composition may contain, for example, the physiologically acceptable polyols described above. The “physiologically acceptable polyols” include glycerin, propylene glycol, polyethylene glycol, dipropylene glycol and diethylene glycol, and preferably is propylene glycol or glycerin. The aqueous suspension or pharmaceutical composition can contain the physiologically acceptable polyol at a concentration of, for example, 0.001 to 10% or 0.01 to 10%, preferably, 0.02 to 5%, for example, 0.03 to 1%, 0.04 to 0.5%, 0.05 to 0.2%. Alternatively, the aqueous suspension or pharmaceutical composition can contain the physiologically acceptable polyol at a concentration of 0.01 to 10 mg/mL, 0.05 to 5 mg/mL or 0.1 to 3 mg/mL.

The aqueous suspension or aqueous pharmaceutical composition does not contain an oil solvent. The oil solvent means a water-insoluble or slightly water-soluble solvent.

Clobetasol propionate contained in the aqueous suspension or aqueous pharmaceutical composition is in the form of nanoparticles. The mean particle diameter of clobetasol propionate nanoparticles may be 300 nm or less, preferably 250 nm or less, 240 nm or less, 230 nm or less, 220 nm or less, 210 nm or less, 200 nm or less, 190 nm or less, 180 nm or less, 170 nm or less, 160 nm or less, 150 nm or less, 140 nm or less, 130 nm or less, 120 nm or less or 110 nm or less. The ranges of mean particle diameter of clobetasol propionate may be, for example, 50 to 300 nm, 50 to 250 nm, 50 to 240 nm, 50 to 230 nm, 50 to 220 nm, 50 to 210 nm, 50 to 200 nm, 50 to 190 nm, 50 to 180 nm, 50 to 170 nm, 50 to 160 nm, 50 to 150 nm, 50 to 140 nm, 50 to 130 nm, 50 to 120 nm, 50 to 110 nm, 100 to 300 nm, 100 to 250 nm, 100 to 240 nm, 100 to 230 nm, 100 to 220 nm, 100 to 210 nm, 100 to 200 nm, 100 to 190 nm, 100 to 180 nm, 100 to 170 nm, 100 to 160 nm, 100 to 150 nm, 100 to 140 nm, 100 to 130 nm, 100 to 120 nm or 100 to 110 nm.

The 90% diameter (D90) of clobetasol propionate nanoparticles contained in the aqueous suspension or aqueous pharmaceutical composition is 450 nm or less, preferably 400 nm or less, 350 nm or less, 300 nm or less, 290 nm or less, 280 nm or less, 270 nm or less, 260 nm or less, 250 nm or less, 240 nm or less or 230 nm or less. The ranges of 90% diameter (D90) of clobetasol propionate may be, for example, 50 to 400 nm, 50 to 350 nm, 50 to 300 nm, 50 to 290 nm, 50 to 280 nm, 50 to 270 nm, 50 to 260 nm, 50 to 250 nm, 50 to 240 nm, 50 to 230 nm, 100 to 400 nm, 100 to 350 nm, 100 to 300 nm, 100 to 290 nm, 100 to 280 nm, 100 to 270 nm, 100 to 260 nm, 100 to 250 nm, 100 to 240 nm or 100 to 230 nm.

The 50% diameter (D50) of clobetasol propionate nanoparticles contained in the aqueous suspension or aqueous pharmaceutical composition may be 200 nm or less, preferably 190 nm or less, 180 nm or less, 170 nm or less, 160 nm or less, 150 nm or less, 140 nm or less, 130 nm or less, 120 nm or less, 110 nm or less or 100 nm or less. The ranges of 50% diameter (D50) of clobetasol propionate may be 50 to 190 nm, 50 to 180 nm, 50 to 170 nm, 50 to 160 nm, 50 to 150 nm, 50 to 140 nm, 50 to 130 nm, 50 to 120 nm, 50 to 110 nm, 50 to 100 nm, 80 to 190 nm, 80 to 180 nm, 80 to 170 nm, 80 to 160 nm, 80 to 150 nm, 80 to 140 nm, 80 to 130 nm, 80 to 120 nm, 80 to 110 nm or 80 to 100 nm.

Clobetasol propionate nanoparticles contained in the aqueous suspension or aqueous pharmaceutical composition may meet two or more particle diameter conditions selected from the mean particle diameter, the 90% diameter (D90) and the 50% diameter (D50) described above. Clobetasol propionate nanoparticles contained in the aqueous suspension can have, for example, a mean particle diameter of 166 nm or less, a D50 of 138 nm or less and/or a D90 of 241 nm or less. Clobetasol propionate nanoparticles contained in the aqueous pharmaceutical composition can have, for example, a mean particle diameter of 204 nm or less, a D50 of 177 nm or less and/or a D90 of 306 nm or less.

Clobetasol propionate contained in the aqueous suspension as the active component is in the form of nanoparticles, which enables the aqueous suspension to be filter-sterilized, and thus the aqueous suspension can be sterilized easily and have minimal effect on the physicochemical properties of the active component.

The nanoparticle of clobetasol propionate contained in the aqueous suspension is preferably produced by mixing clobetasol propionate, a physiologically acceptable salt, a physiologically acceptable polyol and a dispersion stabilizer. More preferably, the nanoparticle of clobetasol propionate is produced by mixing clobetasol propionate, a physiologically acceptable salt, a physiologically acceptable polyol and a dispersion stabilizer, with adding lecithin (e.g., hydrogenated soybean lecithin) during or after pulverization.

The aqueous suspension includes, for example, a preparation containing, nanoparticles of clobetasol propionate; sodium chloride; hydrogenated soybean lecithin; glycerin; anhydrous citric acid; one or more substances selected from POE-POP glycols, polyoxyethylene hydrogenated castor oils, Polysorbate 80, PVA and POE-POP block copolymers; benzalkonium chloride, sorbic acid or salts thereof (potassium sorbate, sodium sorbate and triclocarban sorbate) or paraoxybenzoates (methyl parahydroxybenzoate, ethyl parahydroxybenzoate, propyl parahydroxybenzoate and butyl parahydroxybenzoate); hydroxypropyl methylcellulose and/or methyl cellulose; and sodium citrate (including trisodium citrate).

The aqueous suspension and the pharmaceutical composition can contain water as the main component. The pharmaceutical composition, the aqueous suspension and/or the diluent herein may contain, as necessary, various additives such as a stabilizer, a flavoring agent, a thickener, a surfactant, a preservative, a disinfectant or antibacterial agent, a pH control agent, a tonicity agent and a buffer.

The preservative and the disinfectant or antibacterial agent include sorbic acids or salts thereof (potassium sorbate, sodium sorbate and triclocarban sorbate), paraoxybenzoates (methyl parahydroxybenzoate, ethyl parahydroxybenzoate, propyl parahydroxybenzoate and butyl parahydroxybenzoate), acrinol, methylrosanilinium chloride, benzalkonium chloride, benzethonium chloride, cetylpyridinium chloride, cetylpyridinium bromide, chlorhexidine or salts thereof, polyhexamethylene biguanide, alkylpolyaminoethylglycine, benzyl alcohol, phenethyl alcohol, chlorobutanol, isopropanol, ethanol, phenoxyethanol, silver supported on zirconium phosphate, mercurochrome, povidone iodine, thimerosal, dehydroacetic acid, chloroxylenol, chlorophen, resorcinol, orthophenylphenol, isopropylmethylphenol, thymol, hinokitiol, sulfamine, lysozyme, lactoferrin, triclosan, 8-hydroxyquinoline, undecylenic acid, caprylic acid, propionic acid, benzoic acid, halocarban, thiabendazole, polymyxin B, 5-chloro-2-methyl-4-isothiazolin-3-one, 2-methyl-4-isothiazolin-3-one, polylysine, hydrogen peroxide, polidronium chloride, Glokill (tradename: e.g., Glokill PQ. Rhodia), polydiaryl dimethyl ammonium chloride, poly [oxyethylene (dimethyliminio) ethylene-(dimethyliminio) ethylene dichloride], polyethylene polyamine-dimethylamine epichlorohydrin polycondensates (tradename: e.g., Busan 1157, Buckman Laboratories International, Inc.) and biguanide compounds (Cosmocil CQ (tradename, about 20 wt % content of polyhexamethylenebiguanide hydrochloride, Arch Personal Care Products L.P.)), and pharmacologically acceptable salts thereof. Benzalkonium chloride is preferable.

The pH control agent includes inorganic acids (hydrochloric acid, sulfuric acid, phosphoric acid, polyphosphoric acid and boric acid), organic acids (lactic acid, acetic acid, citric acid, anhydrous citric acid, tartaric acid, malic acid, succinic acid, oxalic acid, gluconic acid, fumaric acid, propionic acid, aspartic acid, epsilon-aminocaproic acid, glutamic acid and aminoethylsulfonic acid), gluconolactone, ammonium acetate, inorganic bases, (sodium hydrogen carbonate, sodium carbonate, potassium hydroxide, sodium hydroxide, calcium hydroxide and magnesium hydroxide), organic bases (monoethanolamine, triethanolamine, diisopropanolamine, triisopropanolamine and lysine), borax, and pharmacologically acceptable salts thereof.

The tonicity agent includes inorganic salts (sodium chloride, potassium chloride, sodium carbonate, sodium hydrogen carbonate, calcium chloride, magnesium sulfate, sodium hydrogen phosphate, disodium hydrogen phosphate, dipotassium hydrogen phosphate, sodium thiosulfate and sodium acetate), polyhydric alcohols (glycerin, propylene glycol, ethylene glycol and 1,3-butylene glycol), saccharides (glucose, mannitol and sorbitol).

The buffer includes tris buffer, borate buffer, phosphate buffer, carbonate buffer, citrate buffer, acetate buffer, epsilon-aminocaproic acid and aspartate. Specific examples include boric acid or salts thereof (sodium borate, potassium tetraborate and potassium metaborate), phosphoric acid or salts thereof (sodium hydrogen phosphate, sodium dihydrogen phosphate and potassium dihydrogen phosphate), carbonic acid or salts thereof (sodium hydrogen carbonate and sodium carbonate), citric acid or salts thereof (sodium citrate, potassium citrate and anhydrous citric acid).

“Thickener” refers to substances that increase the viscosity and/or physical stability of the pharmaceutical composition of the present disclosure. Suitable “thickeners” for the purposes of the present invention include, but are not limited to, stearyl alcohol, carbopol, dimethicone and polymers.

The viscosity of the aqueous suspension and the pharmaceutical composition herein can be 1 to 5 mPa·s, and may be, for example, 1 to 3 mPa·s.

The “%” as used herein in the composition or the content refers to weight % (w/w), unless otherwise stated.

The aqueous suspension containing nanoparticles of clobetasol propionate of the present disclosure has advantages in transparency, dispersibility, storage stability, transferability into the conjunctiva, and transferability into the aqueous humor, with low irritability, and thus is easily sterilized and has good temporal stability and dispersion stability. The suspension can be used for pharmaceutical compositions for parenteral administration, specifically for eye drops.

The nanoparticle of clobetasol propionate can be produced by mixing clobetasol propionate with a physiologically acceptable salt and a physiologically acceptable polyol, and wet pulverizing the organic compound. The production method is described in detail in International Publication No. WO 2008/126797. The mixing step only requires clobetasol propionate; the physiologically acceptable salt and the physiologically acceptable polyol are all mixed together in the end, and the order of addition is not limited. The mixing step may be achieved, for example, by adding the physiologically acceptable salt and the physiologically acceptable polyol to clobetasol propionate or alternatively by adding clobetasol propionate to the physiologically acceptable salt and the physiologically acceptable polyol. Clobetasol propionate nanoparticles contained in a powder of the present disclosure can be produced by adding the physiologically acceptable salt and the physiologically acceptable polyol to an organic compound having a melting point of 80° C. or more, and wet pulverizing the organic compound. In this method, the aqueous suspension can be prepared without removing the salt and the polyol. Since there is no need to remove the salt and the polyol, the suspension can be prepared by very simple steps. The wet pulverization is achieved by mixing the organic compound, the salt and the polyol, and kneading the mixture. The nanoparticle of clobetasol propionate can be preferably produced by adding lecithin during or after the pulverization step.

Clobetasol propionate nanoparticle is produced preferably by wet pulverization without using a hard solid pulverization aid, more preferably without using a solid pulverization aid such as glass products, metallic products such as stainless steel, ceramic products such as zirconia and alumina, and large molecular products such as rigid polystyrene. Most preferably, clobetasol propionate nanoparticle is produced by wet pulverization without using a solid pulverization aid other than the physiologically acceptable salt and the viscosity modifier.

The term “physiologically acceptable” means it is believed not to cause physiological problems when administered into a body. The physiological acceptance of a certain substance is suitably determined depending on the species to be administered as well as modes of administration. Examples of the physiologically acceptable solvent include substances which are approved as additives or solvents for pharmaceutical drugs or for food products.

The “physiologically acceptable salt” herein is not limited as long as it can be administered without causing physiological problems. The physiologically acceptable salt preferably has low solubility to polyols, high solubility to water and/or low moisture absorption with suitable hardness for pulverization of the organic compound. More preferably, the physiologically acceptable salt used in the method for producing clobetasol propionate nanoparticle has two or more of these properties. The solubility of physiologically acceptable salt to polyols is preferably 10% (mass/volume) or less. The physiologically acceptable salt is preferably highly soluble to water for easy removal after pulverization. Specific examples include the salts listed above in this specification.

The “physiologically acceptable salt” is preferably pulverized for adjusting the particle diameter before it is mixed with clobetasol propionate. Moreover, the physiologically acceptable salt may be dried under reduced pressure at a temperature of 30 to 200° C. to reduce a contained water for preventing particle fusion and growth caused by the contained water. When the particle diameter of the physiologically acceptable salt is adjusted in advance, the particle volume mean diameter may be, for example, 5 to 300 μm, 10 to 200 μm, preferably 0.01 to 300 μm, more preferably 0.1 to 100 μm, further preferably 0.5 to 50 μm, most preferably 1 to 5 μm. The amount of salt to be used is preferably 1 to 100 times, more preferably 5 to 30 times, further preferably 10 to 20 times a mass of clobetasol propionate. One kind of salt may be used singly, or two or more kinds of salts may be used together.

The “physiologically acceptable polyol” used in the method for producing clobetasol propionate nanoparticles is not limited as long as it can be administered without causing any physiological problems. The preferable physiologically acceptable polyol has low solubility to salts, high solubility to water, a low freezing point and/or a high ignition point. The physiologically acceptable polyol is preferably highly soluble to water for easy removal after pulverization.

The polyol used in the method for producing clobetasol propionate nanoparticles preferably has a high viscosity. The viscosity of a polyol at 20° C. may be 40 mPa·s or more, preferably 50 mPa·s or more, more preferably 80 mPa·s or more. The upper limit of the viscosity at 20° C. of the polyol to be used in the method for producing clobetasol propionate nanoparticles is not limited, and, for example, can be selected from the ranges from 40 mPa·s or more to 5,000 mPa·s or less, preferably 50 mPa·s or more to 3,000 mPa·s or less, more preferably 80 mPa·s or more to 2,000 mPa·s or less. Specific examples include the polyols listed above in this specification.

The amount of physiologically acceptable polyol used in the method for producing clobetasol propionate nanoparticles is preferably 0.5 to 100 times, more preferably 1 to 10 times a mass of the organic compound to be pulverized. The kind of polyol can be suitably determined according to the solubility of the organic compound to be pulverized. One kind of polyol may be used singly, or two or more kinds of polyols may be used together.

In the method for producing nanoparticles of clobetasol propionate, the kneaded product containing clobetasol propionate, the polyol and the salt preferably has a high viscosity. The viscosity of the kneaded product can be increased preferably by using the mixture in which a viscosity modifier is added to the polyol, or by adding a viscosity modifier independently from the polyol, which can effectively improve the pulverization efficiency. The viscosity modifiers described above can be added to the polyol. The viscosity at 20° C. of a polyol to which the viscosity modifier is added is preferably 1,000 mPa·s or more, more preferably 2,000 mPa·s or more, further preferably 5,000 mPa·s or more, most preferably 10,000 mPa·s or more. The upper limit of the viscosity at 20° C. of the polyol to which the viscosity modifier is added is not limited, and, for example, can be selected from the ranges from 1,000 mPa·s or more to 5,000,000 mPa·s or less, preferably 1,000 mPa·s or more to 1,000,000 mPa·s or less, more preferably 2,000 mPa·s or more to 500,000 mPa·s or less, further preferably 5,000 mPa·s or more to 300,000 mPa·s or less, most preferably 10,000 mPa·s or more to 100,000 mPa·s or less.

In the method for producing clobetasol propionate nanoparticles, any grinder can be used for wet pulverization of clobetasol propionate without limitation, as long as it can mechanically knead and disperse clobetasol propionate, the salt, the polyol and/or the dispersion stabilizer. Examples of the commonly used grinder include kneaders, two rolls, three rolls, fret mills, hoover mullers, and disk blade kneader dispersers.

The pulverization temperature can be suitably determined according to clobetasol propionate to be pulverized and the type of grinder. The pulverization temperature is not limited, and preferably is −50 to 50° C., more preferably −20 to 30° C., and most preferably −10 to 25° C. The pulverization time can be also suitably determined according to the organic compound to be pulverized and the type of grinder. The pulverization time can be, for example, 1 to 50 hours, 2 to 30 hours, 3 to 20 hours, 4 to 18 hours, and 5 to 10 hours.

After completion of the pulverization of clobetasol propionate, the objective particles of pulverized clobetasol propionate can be obtained without removing the salt and the polyol which are used for the pulverization. Because a washing step is unnecessary, nanoparticle preparation is produced more simply at a lower cost. The suspension can be prepared by homogenizing the mixture of clobetasol propionate, the salt, the polyol and/or the viscosity modifier in a solvent using a homogenizer. The solvent used for homogenizing the mixture is not limited as long as it easily dissolves the polyol, the salt and the viscosity modifier while minimally dissolving the pulverized clobetasol propionate and is physiologically acceptable. The solvent is preferably water but any solvent other than water can be used, including a mixture of water and an organic solvent such as acetic acid, methanol or ethanol. The homogenized mixture can be filtered, if necessary. The filtration method is not limited; any known filtration method used for filtering organic compounds contained therein can be employed. Examples of the filtration method include a reduced pressure filtration method, an applied pressure filtration method, and an ultrafiltration membrane method.

The pulverized particles typically have a high surface energy and thus easily agglomerate. Thus, the agglomeration inhibitor described above may be added after removing the salts etc. to prevent secondary agglomeration. One kind of agglomeration inhibitor may be used singly, or two or more kinds of agglomeration inhibitors may be used together.

After the salt and the polyol are removed, the obtained particles of pulverized clobetasol propionate can be dried to remove the solvent used for removing the salt etc. The drying method is not limited; any method commonly used for drying an organic compound can be employed. Examples of the drying method include a reduced pressure drying method, a freeze-drying method, a spray drying method, and a spray-freezing-drying method. The drying temperature and drying time in these drying methods are not limited, but a low temperature is preferable for maintaining the chemical stability of organic compound particles for medical use and preventing secondary particle agglomeration. For the same reason, the methods of freeze-drying, spray drying, and spray-freezing-drying are preferable.

### Pharmaceutical Composition

The present disclosure relates to the pharmaceutical composition containing nanoparticles of clobetasol propionate. The pharmaceutical composition is preferably a pharmaceutical composition for parenteral administrations such as injections or topical preparations. The type of pharmaceutical composition herein is not limited. Examples of the formulation include topical eye preparations (e.g., eye drops). These preparations can be produced in accordance with a conventional method. The pharmaceutical composition preferably contains a dispersion stabilizer.

The pharmaceutical composition may contain a pharmacologically acceptable carrier (an additive for preparations). The kind of additives for preparations used for manufacturing the pharmaceutical composition, the proportion of the additives for preparations relative to the active component, or the method for manufacturing the pharmaceutical composition may be suitably selected by the person skilled in the art depending on the form of composition. The additive for preparations can be an inorganic or organic, or a solid or liquid substance. Specific examples of such substance include lactose, glucose, mannitol, dextrin, cyclodextrin, starch, sucrose, magnesium aluminometasilicate, synthetic aluminum silicate, sodium carboxymethyl cellulose, hydroxypropyl starch, carboxymethyl cellulose calcium, ion exchange resin, methyl cellulose, gelatin, gum arabic, hydroxypropylcellulose, hydroxypropyl methylcellulose, polyvinylpyrrolidone, PVA, light anhydrous silicic acid, magnesium stearate, talc, tragacanth, bentonite, veegum, titanium oxide, fatty acid sorbitan ester, sodium lauryl sulfate, glycerin, fatty acid glycerin ester, purified lanolin, glycerogelatin, polysorbate, Macrogol, vegetable oil, wax, liquid paraffin, white petrolatum, fluorocarbon, nonionic surfactant, propylene glycol, water, benzalkonium chloride, hydrochloric acid, sodium chloride, sodium hydroxide, lactic acid, sodium, sodium monohydrogen phosphate, sodium dihydrogen phosphate, citric acid, sodium citrate, disodium edetate, Poloxamer 407 and polycarbophil. For example, the pharmaceutical composition may contain one or more additives for preparations selected from POE-POP glycol, PVA, hydroxypropyl methylcellulose and methyl cellulose.

In one embodiment, the present disclosure provides the method for preparing an aqueous pharmaceutical composition containing clobetasol propionate nanoparticles, comprising mixing a diluent and the aqueous suspension containing clobetasol propionate nanoparticles.

In preparing the pharmaceutical composition (e.g., topical eye preparations (preferably eye drops)), the pH and osmotic pressure are not limited as long as they are acceptable for the topical preparations, and preferably is pH 3 to 9.5, more preferably is pH 3.5 to 9, further preferably is pH 4 to 8. The ratio of osmotic pressure of the preparation (except ointments) to saline is, for example, about 0.3 to 4.3, preferably about 0.3 to 2.2, particularly preferably about 0.5 to 1.5. The pH and osmotic pressure can be controlled using a pH control agent, a tonicity agent or salts by a method known in the art.

The pharmaceutical composition can be suitably produced by a known method, for example, by mixing the aqueous suspension containing clobetasol propionate nanoparticles with desired components in a suitable diluent such as distilled water or purified water, adjusting the above osmotic pressure and pH, subjecting the composition to high pressure steam sterilization or filter-sterilization under aseptic conditions, and filling it aseptically into a washed sterilized container.

### Method of Treating or Ameliorating an Operative Complication of a Cataract Surgery

The present disclosure provides a method of treating or ameliorating an operative complication of a cataract surgery in a subject in need thereof comprising administering a pharmaceutical composition comprising the aqueous suspension to a subject in need thereof in a therapeutically effective amount.

Examples of the operative complication include but are not limited to high anterior chamber cell (ACC) count, pain, impaired visual acuity, ocular inflammation and ocular infection.

In one embodiment of the disclosure, the cataract surgery is an uncomplicated cataract surgery or complicated cataract surgery. In one embodiment of the disclosure, the cataract surgery is an uncomplicated cataract surgery.

In some embodiments of the disclosure, the aqueous suspension comprises 0.01 wt % to 0.2 wt %; 0.015 wt % to 0.18 wt %; 0.02 wt % to 0.16 wt %; 0.025 wt % to 0.14 wt %; 0.03 wt % to 0.12 wt %; 0.035 wt % to 0.1 wt %; 0.04 wt % to 0.08 wt %; 0.04 wt % to 0.06 wt %; 0.05 wt %; 0.1 wt % of clobetasol propionate. In some further embodiments of the disclosure, the aqueous suspension comprises 0.05 wt % or 0.1% wt of clobetasol propionate.

In some embodiments of the disclosure, the pharmaceutical composition is administered before the cataract surgery, in progress of the cataract surgery or after the cataract surgery. In one embodiment of the disclosure, wherein the pharmaceutical composition is administered after the cataract surgery.

The application amount can be suitably determined depending on conditions, and is typically applied to the eye once to six times a day, for example, once, twice, three times, four times, five times or six times a day, with about 1 to 3 drops per application. In some embodiments of the disclosure, the pharmaceutical composition is administered once, twice, three times, or four times a day. In some further embodiments of the disclosure, the pharmaceutical composition is administered twice (BID) or once daily (QD).

In one embodiment of the disclosure, the pharmaceutical composition is administered for at least once. In one embodiment of the disclosure, the pharmaceutical composition is administered on the first post-operative day (POD 1). In one embodiment of the disclosure, the pharmaceutical composition is administered on the day of the cataract surgery.

In one embodiment of the disclosure, the pharmaceutical composition is administered for at least one day. The administration period can be any period of time until symptoms adequately subside, for example, two weeks to one year. In some embodiments of the disclosure, the pharmaceutical composition is administered for 3 days to 28 days; 4 days to 27 days; 5 days to 26 days; 6 days to 26 days; 7 days to 25 days; 8 days to 24 days; 9 days to 23 days; 10 days to 22 days; 11 days to 21 days; 12 days to 20 days; 13 days to 19 days; 14 days to 18 days; 15 days to 17 days. In some further embodiments of the disclosure, the pharmaceutical composition is administered for 7 days, 14 days, 21 days or 28 days.

The dose and number of administrations of the pharmaceutical composition are not limited and can be suitably selected at a physician's discretion depending on purpose of prevention of deterioration/progress and/or the purpose of treatment of the disease to be treated, the type of disease, and patient's conditions such as body weight and age. The dose is generally about 0.01 to 1000 mg (on an active component weight basis) a day for an adult, and can be administered once or several times a day.

When the aqueous pharmaceutical composition of the present disclosure is for topical administration, it is directly administered to a topical area such as an affected site, an area around an affected site, or an organ including an affected site. When the pharmaceutical composition of the present disclosure is a topically administrable preparation, it is applicable every day or any number of times after the cataract surgery is performed.

### Ophthalmic Composition

The present disclosure provides an ophthalmic composition comprising: nanoparticles of clobetasol propionate, wherein a mean particle diameter of the nanoparticles is 300 nm or less and a D90 particle diameter of the nanoparticles is 450 nm or less;

a physiologically acceptable salt;

glycerin;

hydrogenated soybean lecithin;

anhydrous citric acid;

boric acid;

EDTA; and
BAK,
   wherein the concentration of benzalkonium chloride is lower than 45 ppm, 44 ppm, 43 ppm, 42 ppm, 41 ppm, 40 ppm, 39 ppm, 38 ppm, 37 ppm, 36 ppm, 35 ppm, 34 ppm, 33 ppm, 32 ppm, 31 ppm, 30 ppm, 29 ppm, 28 ppm, 27 ppm, 26 ppm, 25 ppm, 24 ppm, 23 ppm, 22 ppm, 21 ppm, 20 ppm, 19 ppm, 18 ppm, 17 ppm, 16 ppm, or 15 ppm. In some embodiments of the disclosure, the concentration of benzalkonium chloride is from 15 ppm to 45 ppm.

In some embodiments of the disclosure, by using EDTA and boric acid, the BAK concentration is reduced, such as from 60 ppm, to lower than 45 ppm. Not willing limited by theory, it is believed that lower BAK concentration is desirable because of the negative effects on cornea and eye health in general.

In some embodiments of the disclosure, the ophthalmic composition comprises the nanoparticles of clobetasol propionate, sodium chloride, hydrogenated soybean lecithin, glycerin, anhydrous citric acid, polyvinyl alcohol, Poloxamer 407, BAK, methylcellulose, boric acid, and EDTA.

In some embodiments of the disclosure, the pH value is reduced from 7 to 6 compared to the conventional products. Not willing limited by theory, it is believed that the lower pH retards and/or reduces the formation of impurities. In some embodiments, increasing methylcellulose content also helps retention time on the eye by increasing viscosity.

In some embodiments of the disclosure, the ophthalmic composition comprises 0.05 wt % to 0.15 wt % of the nanoparticles of clobetasol propionate, 0.5 wt % to 1.5 wt % sodium chloride, 0.05 wt % to 0.15 wt % hydrogenated soybean lecithin, 0.1 wt % to 0.4 wt % glycerin, 0.003 wt % to 0.01 wt % anhydrous citric acid, 0.05 wt % to 0.15 wt % polyvinyl alcohol, 0.002 wt % to 0.01 wt % Poloxamer 407, 0.003 wt % to 0.004 wt % BAK, 0.3 wt % to 0.8 wt % methylcellulose, 0.15 wt % to 0.35 wt % boric acid, and 0.01 wt % to 0.07 wt % EDTA.

The following examples are provided to aid those skilled in the art in practicing the present disclosure.

### Examples

Preparation of a 0.05% Ophthalmic Nanoparticle Suspension (Mean Particle Diameter of about 130 nm) (Suspension 1)

To a 5.0 L water-cooling vertical mixer (INOUE MFG., INC.), 50 g of clobetasol propionate micronized (melting point: 190.0 to 200.0° C., FARMABIOS SpA) having a mean volume weighted mean D [4,3] (De Brouckere mean) particle diameter of 2.315 mm, 550 g of sodium chloride (Tomita Salt K-30, Tomita Pharmaceutical Co., Ltd.), 50 g of hydrogenated soybean lecithin (Phospholipon 90H, Lipoid GmbH) and 4 g of anhydrous citric acid (Sigma-Aldrich Co. LLC.) were charged, and homogeneously mixed. To the mixture, 75 g of glycerin (Sigma-Aldrich Co. LLC.) was added while keeping the mixture in a state of dough (Nanomixture) and pulverized at 4±2° C. for 6 hours.

The particle size distribution of the nanoparticle was measured as follows. 0.1 g of the obtained pulverized-kneaded product (dough (Nanomixture)) and 5 g of 0.01% Poloxamer 407 solution (prepared from Poloxamer 407, BASF SE) as the dispersant were weighed into a 50 mL screw bottle, and dispersed homogeneously using an ultrasonic device (Model VS-100III, AS ONE Corporation), and 45 g of 0.01% Poloxamer 407 aqueous solution was added thereto to obtain 50 g of a suspension. The obtained suspension was measured for particle size distributions using a particle size distribution analyzer (Model ELSZ-2000ZS, Otsuka Electronics Co., Ltd.), and the particle size distributions of clobetasol propionate were found to have a mean particle diameter of 133 nm, a 10% particle diameter (D10) of 75 nm, a particle size distribution (D50) of 116 nm and a 90% particle diameter (D90) of 184 nm.

320 g of the pulverized-kneaded product (dough (Nanomixture)) was mixed with 21.075 kg of 0.01% Poloxamer 407 solution and dispersed by L5M-A high-shear homogenizer then transfer into a 50 L jacketed stainless steel tank, mixed with 4.2 kg of a 1.0% PVA aqueous solution (prepared from Polyvinyl Alcohol, Merck KGaA) to give a crude dispersion, which was processed using a high pressure homogenizer (H-20, SANWA ENGINEERING LTD.) to obtain a dispersion. To 18.75 kg (Total dispersion amount is 25.6 kg) of dispersion, 101.8 g of 1% benzalkonium chloride (BAK) aqueous solution, 6 kg of a 2.5% Methylcellulose (MC) with 1.25% Boric acid (BA) aqueous solution and 300 g of 5% EDTA-2Na 2H₂O aqueous solution were added, to which subsequently added 200 g of 1 M tri-sodium citrate aqueous solution and about 85 g glycerin were added for pH and osmolality adjustments. Water for injection was then added thereto to give a total amount of 30 kg to obtain a 0.05% ophthalmic nanosuspension (a mean particle diameter of 134 nm). The obtained ophthalmic suspension had pH 6 and osmolality 285 mOsmol/kg. Following, re-dispersion, sterile filtration and filling were conducted to obtain 0.05% clobetasol propionate ophthalmic nanosuspension.

Table 1 shows the composition of each of the 0.05% clobetasol propionate opthhalmic nanosuspensions.

| Component | Composition (%) |
|---|---|
| Clobetasol propionate | 0.054 |
| Sodium chloride | 0.59 |
| Hydrogenated soybean lecithin | 0.054 |
| Glycerin | 0.37 |
| Anhydrous citric acid | 0.004 |
| PVA | 0.10 |
| Poloxamer 407 | 0.005 |
| Benzalkonium chloride (BAK) | 0.0036 |
| Methylcellulose | 0.5 |
| Boric Acid | 0.25 |
| EDTA-2Na 2H₂O | 0.05 |
| Tri-Sodium citrate | Quantum sufficit |
| Water for injection | Quantum sufficit |

Preparation of a 0.05% Placebo Ophthalmic Nanoparticle Suspension (Mean Particle Diameter of about 130 nm) (Placebo Suspension 1)

To a 5.0 L water-cooling vertical mixer (INQUE MFG., INC.), 100 g of hydrogenated soybean lecithin (Phospholipon 90H, Lipoid GmbH), 550 g of sodium chloride (Tomita Salt K-30, Tomita Pharmaceutical Co., Ltd.) and 4 g of anhydrous citric acid (Sigma-Aldrich Co. LLC.) were charged, and homogeneously mixed. To the mixture, 75 g of glycerin (Sigma-Aldrich Co. LLC.) was added while keeping the mixture in a state of dough (Nanomixture) and pulverized at 4±2° C. for 6 hours.

The particle size distribution of the nanoparticle was measured as described in “Suspension 1.” The particle size distributions were found to have a mean particle diameter of 98 nm, a 10% particle diameter (D10) of 43 nm, a particle size distribution (D50) of 78 nm and a 90% particle diameter (D90) of 158 nm.

320 g of the pulverized-kneaded product (dough (Nanomixture)) was mixed with 21.075 kg of 0.01% Poloxamer 407 solution and dispersed by L5M-A high-shear homogenizer, and then transferred into a 50 L jacketed stainless steel tank, mixed with 4.2 kg of a 1.0% PVA (Merck KGaA) aqueous solution to give a crude dispersion, which was processed using a high pressure homogenizer (H-20, SANWA ENGINEERING LTD.) to obtain a dispersion. To 18.75 kg (Total dispersion amount is 25.6 kg) dispersion, 101.8 g of 1% benzalkonium chloride (BAK) aqueous solution, 6 kg of a 2.5% Methylcellulose (MC) with 1.25% Boric acid (BA) aqueous solution and 300 g 5% EDTA-2Na 2H₂O aqueous solution were added, to which subsequently 200 g of 1M tri-sodium citrate and about 85 g glycerin was added to adjust pH and osmolality. Water for injection was then added thereto to give a total amount of 30 kg to obtain a 0.05% placebo ophthalmic nanosuspension (a mean particle diameter of 128 nm). The obtained ophthalmic suspension had pH 6 and osmolality 283 mOsmol/kg. Following, re-dispersion, sterile filtration and filling were conducted to obtain 0.05% placebo ophthalmic nanosuspension.

Table 2 shows the composition of each of the 0.05% placebo clobetasol propionate opthalmic nanosuspensions.

| Component | Composition (%) |
|---|---|
| Clobetasol propionate | 0 |
| Hydrogenated soybean lecithin | 0.11 |
| Sodium chloride | 0.59 |
| Glycerin | 0.37 |
| Anhydrous citric acid | 0.004 |
| PVA | 0.10 |
| Poloxamer 407 | 0.005 |
| Benzalkonium chloride (BAK) | 0.0036 |
| Methylcellulose | 0.5 |
| Boric Acid | 0.25 |
| EDTA-2Na 2H2O | 0.05 |
| Tri-Sodium citrate | Quantum sufficit |
| Water for injection | Quantum sufficit |

Preparation of a 0.1% Ophthalmic Nanoparticle Suspension (Mean Particle Diameter of about 120 nm) (Suspension 2)

To a 5.0 L water-cooling vertical mixer (INOUE MFG., INC.), 50 g of clobetasol propionate micronized (melting point: 190.0 to 200.0° C., FARMABIOS SpA.) having volume weighted mean D [4,3] (De Brouckere mean) particle diameter of 2.315 μm, 550 g of sodium chloride (Tomita Salt K-30, Tomita Pharmaceutical Co., Ltd.), 50 g of hydrogenated soybean lecithin (Phospholipon 90H, Lipoid GmbH) and 4 g of anhydrous citric acid (Sigma-Aldrich Co. LLC.) were charged, and homogeneously mixed. To the mixture, 75 g of glycerin (Sigma-Aldrich Co. LLC.) was added while keeping the mixture in a state of dough (Nanomixture) and pulverized at 4±2° C. for 6 hours.

The particle size distribution of the nanoparticle was measured as described in “Suspension 1.” The particle size distributions of clobetasol propionate were found to have a mean particle diameter of 121 nm, a 10% particle diameter (D10) of 65 nm, a particle size distribution (D50) of 103 nm and a 90% particle diameter (D90) of 171 nm.

666 g of the pulverized-kneaded product (dough (Nanomixture)) was mixed with 21.075 kg of 0.01% Poloxamer 407 solution and dispersed by L5M-A high-shear homogenizer then transfer into a 50 L jacketed stainless steel tank mixed with 4.2 kg of a 1.0% PVA aqueous solution (prepared from polyvinyl alcohol, Merck KGaA) to give a crude dispersion, which was processed using a high pressure homogenizer (H-20, SANWA ENGINEERING LTD.) to obtain a dispersion. To 18.75 kg (Total dispersion amount is 25.94 kg) of dispersion, 101.8 g of 1% benzalkonium chloride (BAK) aqueous solution, 6 kg of a 2.5% Methylcellulose (MC) with 1.25% Boric acid (BA) aqueous solution and 300 g of 5% EDTA-2Na 2H2O were added, to which subsequently a 1 M tri-sodium citrate was gradually added up to pH 6. Water for injection was then added thereto to give a total amount of 30 kg to obtain a 0.1% ophthalmic nanosuspension (a mean particle diameter of 137 nm). The obtained ophthalmic suspension had an Osmolality of 474 mOsmol/kg. Following, re-dispersion, sterile filtration and filling were conducted to obtain 0.1% clobetasol propionate ophthalmic nanosuspension.

Table 3 shows the composition of each of the 0.1% clobetasol propionate opthalmic nanosuspensions.

| Component | Composition (%) |
|---|---|
| Clobetasol propionate | 0.11 |
| Sodium chloride | 1.2 |
| Hydrogenated soybean lecithin | 0.11 |
| Glycerin | 0.17 |
| Anhydrous citric acid | 0.008 |
| PVA | 0.10 |
| Poloxamer 407 | 0.005 |
| Benzalkonium chloride (BAK) | 0.0036 |
| Methylcellulose | 0.5 |
| Boric Acid | 0.25 |
| EDTA-2Na 2H₂O | 0.05 |
| Tri-Sodium citrate | Quantum sufficit |
| Water for injection | Quantum sufficit |

Preparation of a 0.1% Placebo Ophthalmic Nanoparticle Suspension (Mean Particle Diameter of about 110 nm) (Placebo Suspension 2)

To a 5.0 L water-cooling vertical mixer (INOUE MFG., INC.), 100 g of hydrogenated soybean lecithin (Phospholipon 90H, Lipoid GmbH), 550 g of sodium chloride (Tomita Salt K-30, Tomita Pharmaceutical Co., Ltd.) and 4 g of anhydrous citric acid Sigma-Aldrich Co. LLC.) were charged, and homogeneously mixed. To the mixture, 75 g of glycerin (Sigma-Aldrich Co. LLC.) was added while keeping the mixture in a state of dough (Nanomixture) and pulverized at 4±2° C. for 6 hours.

The particle size distribution of the nanoparticle was measured as described in “Suspension 1.” The particle size distributions were found to have a mean particle diameter of 112 nm, a 10% particle diameter (D10) of 50 nm, a particle size distribution (D50) of 92 nm and a 90% particle diameter (D90) of 175 nm.

666 g of the pulverized-kneaded product (dough (Nanomixture)) was mixed with 21.075 kg of 0.01% Poloxamer 407 solution and dispersed by L5M-A high-shear homogenizer then transfer into a 50 L jacketed stainless steel tank mixed with 4.2 kg of a 1.0% PVA aqueous solution (prepared from polyvinyl alcohol, Merck KGaA) to give a crude dispersion, which was processed using a high pressure homogenizer (H-20, SANWA ENGINEERING LTD.) to obtain a dispersion. To the 18.75 kg (Total dispersion amount is 25.94 kg) of dispersion, 101.8 g of 1% benzalkonium chloride (BAK) aqueous solution, 6 kg of a 2.5% Methylcellulose (MC) with 1.25% Boric acid (BA) aqueous solution and 300 g of 5% EDTA-2Na 2H2O aqueous solution were added, to which subsequently a 1 M tri-sodium citrate was gradually added up to pH 6. Water for injection was then added thereto to give a total amount of 30 kg to obtain a 0.1% ophthalmic nanosuspension (a mean particle diameter of 128 nm). The obtained ophthalmic suspension had an osmolality of 473 mOsmol/kg. Following, re-dispersion, sterile filtration and filling were conducted to obtain 0.1% placebo ophthalmic nanosuspension.

Table 4 shows the composition of each of the 0.1% placebo clobetasol propionate opthalmic nanosuspensions.

| Component | Composition (%) |
|---|---|
| Clobetasol propionate | 0 |
| Hydrogenated soybean lecithin | 0.22 |
| Sodium chloride | 1.2 |
| Glycerin | 0.17 |
| Anhydrous citric acid | 0.008 |
| PVA | 0.10 |
| Poloxamer 407 | 0.005 |
| Benzalkonium chloride (BAK) | 0.0036 |
| Methylcellulose | 0.5 |
| Boric Acid | 0.25 |
| EDTA-2Na 2H₂O | 0.05 |
| Tri-Sodium citrate | Quantum sufficit |
| Water for injection | Quantum sufficit |

### METHOD FOR TREATING OPERATIVE COMPLICATIONS OF A CATARACT SURGERY

132 participants (subjects) aged 50 years and older were recruited.

Part A: Subjects with ocular inflammation on the first post-operative day (POD 1) following routine, uncomplicated cataract surgery were randomized to receive 1 drop of Suspension 1 (N=22) or matching Placebo Suspension 1 (N=23) BID (morning and evening) in the study eye for 21 days. Evaluations were performed on PODs 1 (baseline), (4, 8, 15, and 22).

Part B: Subjects were randomized to Suspension 1 (N=22), Suspension 2 (N=22) or matching placebos (N=22 for Placebo Suspension 1 and N=21 for Placebo Suspension 2 for 3 days BID, followed by QD dosing for 11 days Evaluations were performed on PODs 1 (baseline), (4, 8, 15 and 22).

### Anterior Chamber Cell (ACC) Count

### Absolute Anterior Chamber Cell Count

Time Frame: Post-operative Day 22 (Part A or B)

The number of cells in the anterior chamber of the eye was counted using slit-lamp biomicroscopy at each study visit and the results are reported as number of cells in a 1 mm×1 mm area.

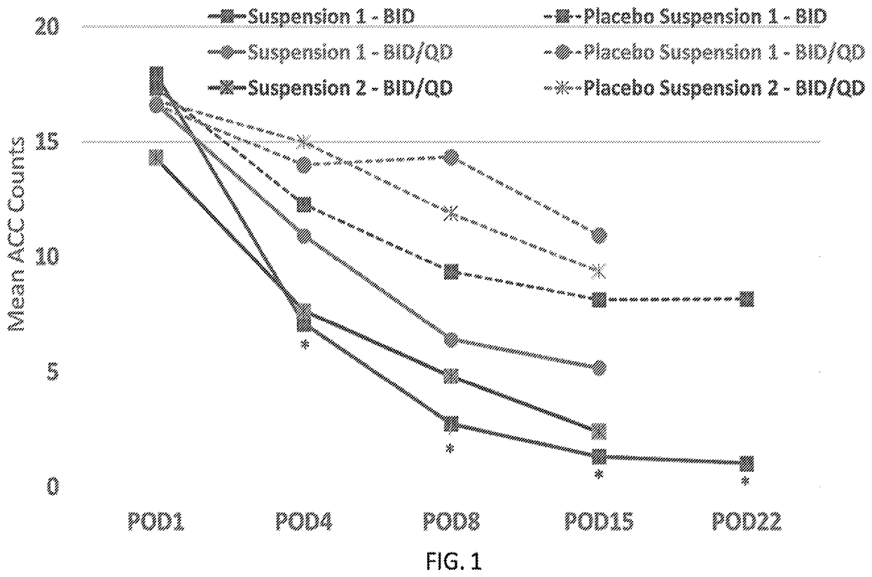

The meaning of ACC counts refers to the number of dead cells. The smaller the value, the lower the number of necrotic cells. The results are shown in FIG. 1. The group of Suspension 1 BID on POD22 exhibits the lowest ACC counts.

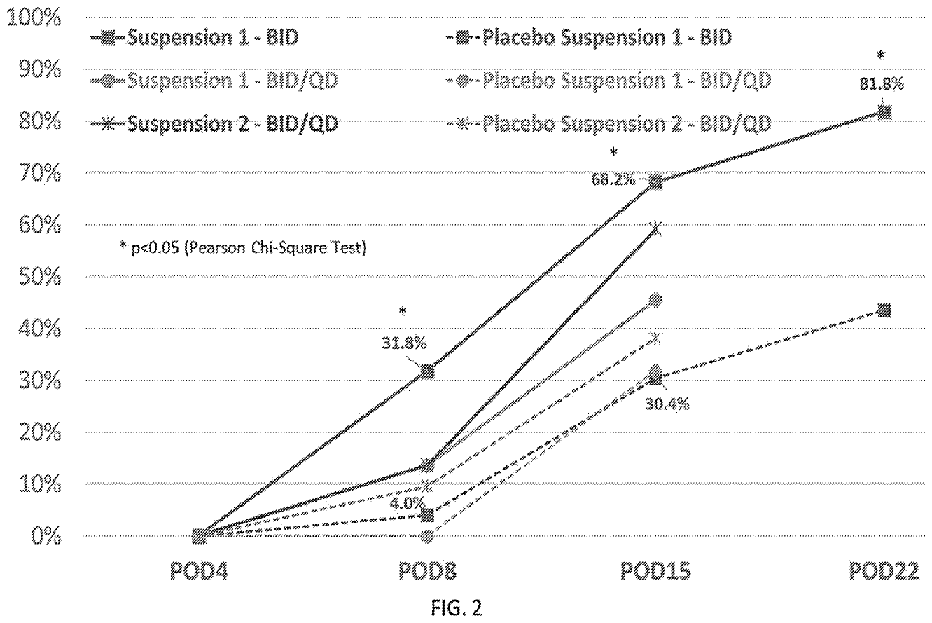

FIG. 2 shows the percentage of subjects with an ACC count of 0 (no cell necrosis) at a given time point. On POD22, Suspension 1 BID group shows no necrosis in about 80% of the subjects' anterior cells.

### Ocular Pain Grade

Time Frame: Post-operative Day 22 (Part A or B)

Ocular pain is measured in each eye using a subject-reported five-point (0-4) ocular pain grade scale (0=no pain; 4=severe pain).

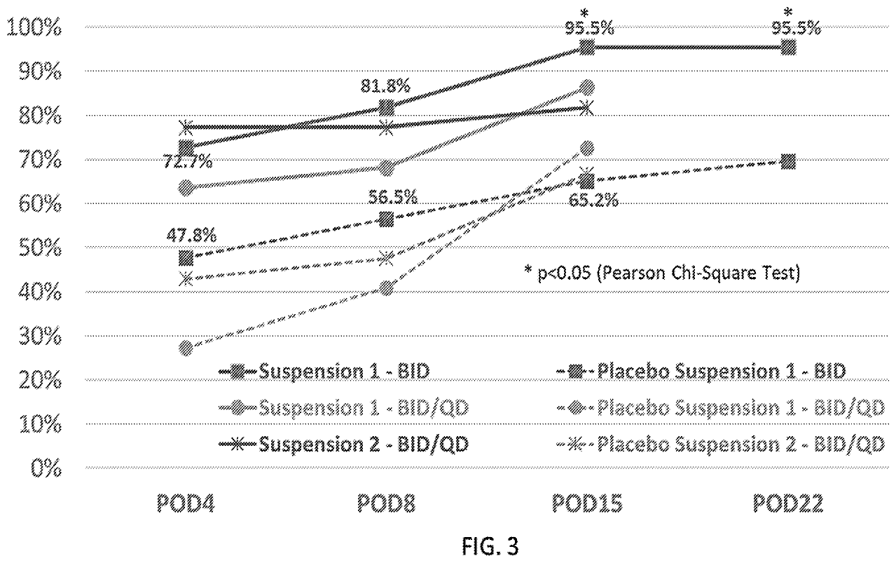

The results are shown in FIG. 3. FIG. 3 shows the percentage of the subjects with a pain index of 0 at each time point. Compared to the placebo group, all Suspension groups demonstrate significantly better effects. On POD22, 90% of the subjects in Suspension 1 BID group have no pain.

### Formulation

20 g of the pulverized-kneaded product (dough (Nanomixture)) was mixed with 1.32 kg of 0.01% Poloxamer 407 solution and dispersed by L5M-A high-shear homogenizer then transfer into a 1 L jacketed stainless steel tank, mixed with 265 g of a 1.0% PVA aqueous solution (prepared from Polyvinyl Alcohol, Merck KGaA) to give a crude dispersion, which was processed using a high pressure homogenizer (L01-YH1, SANWA ENGINEERING LTD.) to obtain a dispersion. To 1.25 kg (Total dispersion amount is 1.61 kg) of dispersion, 0.082 g, 0.062 g. 0.056 g. 0.046 g, or 0.032 g of 10% benzalkonium chloride (BAK) aqueous solution, 400 g of a 2.5% Methylcellulose (MC) with 1.25% Boric acid (BA) aqueous solution and 20 g of 5% EDTA-2Na·2H₂O aqueous solution were added, to which subsequently added 13.35 g of 1 M tri-sodium citrate aqueous solution and about 5.65 g glycerin were added for pH and osmolality adjustments. Water for injection was then added thereto to give a total amount of 2 kg to obtain a 0.05% ophthalmic nanosuspension. The obtained ophthalmic suspension had pH 5.7-6.3 and osmolality 265-270 mOsmol/kg. Following, re-dispersion, sterile filtration and filling were conducted to obtain 0.05% clobetasol propionate ophthalmic nanosuspension.

Table 5 shows the composition of each of the 0.05% clobetasol propionate opthalmic nanosuspensions.

| Component | Composition (%) | | | | |
|---|---|---|---|---|---|
| Clobetasol propionate | 0.054 | | | | |
| Sodium chloride | 0.59 | | | | |
| Hydrogenated soybean | 0.054 | | | | |
| lecithin | | | | | |
| Glycerin | 0.34 | | | | |
| Anhydrous citric acid | 0.004 | | | | |
| PVA | 0.10 | | | | |
| Poloxamer 407 | 0.005 | | | | |
| Benzalkonium chloride | 0.0041 | 0.0031 | 0.0028 | 0.0023 | 0.0016 |
| (BAK) | | | | | |

| | |
|---|---|
| Methylcellulose | 0.5 |
| Boric Acid | 0.25 |
| EDTA-2Na 2H₂O | 0.05 |
| Tri-Sodium citrate | Quantum sufficit |
| Water for injection | Quantum sufficit |

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

1. A method of treating or ameliorating an operative complication of a cataract surgery in a subject in need thereof, said method comprising administering a pharmaceutical composition comprising an aqueous suspension to a subject in need thereof in a therapeutically effective amount, wherein the aqueous suspension comprises:
nanoparticles of clobetasol propionate, wherein a mean particle diameter of the nanoparticles is 300 nm or less and a D90 particle diameter of the nanoparticles is 450 nm or less;
a physiologically acceptable salt;
glycerin;
hydrogenated soybean lecithin; and
anhydrous citric acid; and
wherein the operative complication comprises high anterior chamber cell (ACC) count or pain.

2. The method of claim 1, wherein the pharmaceutical composition is administered before the cataract surgery, in progress of the cataract surgery or after the cataract surgery.

3. The method of claim 1, wherein the operative complication further comprises impaired visual acuity, ocular inflammation and/or ocular infection.

4. The method of claim 1, wherein the cataract surgery is an uncomplicated cataract surgery.

5. The method of claim 1, wherein the aqueous suspension comprises 0.01 wt % to 0.2 wt % of clobetasol propionate.

6. The method of claim 1, wherein the pharmaceutical composition is administered once, twice, three times, or four times a day.

7. The method of claim 1, wherein the pharmaceutical composition is administered on the first post-operative day (POD 1).

8. The method of claim 1, wherein the pharmaceutical composition is administered for at least once.

9. The method of claim 1, wherein the pharmaceutical composition is administered for 3 days to 28 days.

10. The method of claim 1, wherein the nanoparticles are produced by mixing clobetasol propionate, the physiologically acceptable salt, glycerin, hydrogenated soybean lecithin and anhydrous citric acid.

11. The method of claim 10, wherein the nanoparticles are produced by further mixing clobetasol propionate, the physiologically acceptable salt, glycerin, hydrogenated soybean lecithin and anhydrous citric acid with a surface modifier.

12. The method of claim 11, wherein the surface modifier comprises a surfactant, agglomeration inhibitor, viscosity modifier and/or dispersion stabilizer.

13. The method of claim 12, wherein the surface modifier is the dispersion stabilizer.

14. The method of claim 13, wherein the dispersion stabilizer is polyoxyethylene polyoxypropylene glycol and/or polyvinyl alcohol.

15. The method of claim 11, wherein the surface modifier is the viscosity modifier.

16. The method of claim 14, wherein the viscosity modifier is one or more substances selected from the group consisting of methyl cellulose, hydroxypropyl methylcellulose and polyvinyl alcohol.

17. The method of claim 14, wherein the aqueous suspension comprises 1 to 10 mg/mL of the viscosity modifier.

18. The method of claim 1, wherein the pharmaceutical composition is administered as an eye drop.

19. An ophthalmic composition comprising:
nanoparticles of clobetasol propionate, wherein a mean particle diameter of the nanoparticles is 300 nm or less and a D90 particle diameter of the nanoparticles is 450 nm or less;
a physiologically acceptable salt;
glycerin;
hydrogenated soybean lecithin;
anhydrous citric acid;
boric acid;
ethylenediaminetetraacetic acid (EDTA); and
benzalkonium chloride (BAK),
wherein the concentration of benzalkonium chloride is lower than 45 ppm.

20. The ophthalmic composition of claim 19, which comprises the nanoparticles of clobetasol propionate, sodium chloride, hydrogenated soybean lecithin, glycerin, anhydrous citric acid, polyvinyl alcohol, Poloxamer 407, BAK, methylcellulose, boric acid, and EDTA.

21. The ophthalmic composition of claim 19, which comprises 0.05 wt % to 0.15 wt % of the nanoparticles of clobetasol propionate, 0.5 wt % to 1.5 wt % sodium chloride, 0.05 wt % to 0.15 wt % hydrogenated soybean lecithin, 0.1 wt % to 0.4 wt % glycerin, 0.003 wt % to 0.01 wt % anhydrous citric acid, 0.05 wt % to 0.15 wt % polyvinyl alcohol, 0.002 wt % to 0.01 wt % Poloxamer 407, 0.003 wt % to 0.004 wt % BAK, 0.3 wt % to 0.8 wt % methylcellulose, 0.15 wt % to 0.35 wt % boric acid, and 0.01 wt % to 0.07 wt % EDTA.
